(19) United States
(12) Patent Application Publication
(10) Pub. No.: US 2004/0092805 A1
(43) Pub. Date: May 13, 2004

(54) Signal processing method, and pulse photometer using the method

(75) Inventor: Masaru Yarita, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: NIHON KOHDEN CORPORATION

(21) Appl. No.: 10/695,907

(22) Filed: Oct. 30, 2003

(30) Foreign Application Priority Data
Oct. 31, 2002 (JP) P2002-318278
Dec. 13, 2002 (JP) P2002-362261
Sep. 25, 2003 (JP) P2003-333613

(51) Int. Cl. A61B 5/00

(52) U.S. Cl. 600/310

(57) ABSTRACT

A pulse photometer adapted to observe a pulse wave of a living body is disclosed. A light emitter is adapted to irradiate the living body with a first light beam having a first wavelength and a second light beam having a second wavelength which is different from the first wavelength. A converter is operable to convert the first light beam and the second light beam, which have been reflected or transmitted from the living body, into a first data set corresponding to the first wavelength and a second data set corresponding to the second wavelength. A processor is operable to process the first data set and the second data set with a rotating matrix to separate a signal component and a noise component contained in the pulse wave.

### BACKGROUND OF THE INVENTION

The present invention relates to a signal processing method for extracting a biological signal component by processing two types of signals that have been extracted from a single medium substantially at the same time, and more particularly, to an improvement in signal processing in a pulse photometer used in. the medical field, especially in diagnosis of a circulatory organ.

Various methods have already been proposed for separating a signal component and a noise component from two signals that have been extracted from a single medium substantially at the same time.

The methods are usually implemented through frequency domain processing and time domain processing.

Known pulse photometers used in the medical field include an apparatus called a photoplethysmograph, which measures a pulse waveform and a pulse rate; an oxygen saturation SpO₂ measurement apparatus for measuring the concentration of a light-absorbing material included in the blood; an apparatus for measuring the concentration of abnormal hemoglobin, such as carboxyhemoglobin (COHb) or methemoglobin (MetHb); and an apparatus for measuring the concentration of injected dye.

The apparatus for measuring oxygen saturation SpO₂ is particularly called a pulse oximeter.

The principle of the pulse photometer is to determine the concentration of a material of interest from a pulse wave data signal, wherein the data signal is obtained by causing light rays, which exhibit different light absorbances against the material of interest and have a plurality of wavelengths, to transmit through or reflect off a living tissue, and by consecutively measuring the quantity of the transmitted or the reflected light.

If noise is mixed into the pulse wave data, correct computation of a concentration will not be carried out, which may in turn cause erroneous treatment.

In the field of the pulse photometer, attention has been paid to a signal component obtained by dividing a frequency band in order to reduce noise to a lower level, and a method for determining a correlation between two signals has been proposed. However, the method presents a problem of analysis that is very time consuming.

To solve such problems, Japanese Patent No. 3270917 discloses a related-art method of determining oxygen saturation in arterial blood or the concentration of a light-absorbing material. Specifically, a living tissue is exposed to light rays having two different wavelengths, and two pulse wave signals are obtained from the resultant transmitted light. A graph is formed by plotting the magnitudes of the pulse wave signals on the vertical and horizontal axes, to thereby determine a regression line. The oxygen saturation in arterial blood or the concentration of light-absorbing material is determined from the slope of the regression line.

According to this related-art the accuracy of measurement is improved, and power consumption is reduced. However, much computing operation is required to determine a regression line and the slope thereof through use of numerous sampled data sets pertaining to pulse wave signals of the two wavelengths.

Further, Japanese Patent Publication No. 2003-135434A discloses a related-art method of filtering a pulse wave signal through use of frequency analysis, wherein a pulse wave signal is not extracted during the analysis, but a fundamental frequency of a pulse wave signal is determined, and the pulse wave signal is then filtered through use of a filter using a harmonic wave frequency, in order to enhance precision to a much greater extent However, further improvement in determination of a fundamental frequency is desired.

### SUMMARY OF THE INVENTION

It is therefore an object of the invention to provide a signal processing method which alleviates a computing burden required to process two types of signals (e.g., pulse wave signals) extracted substantially simultaneously from a single medium (e.g., living body) to thus extract a common signal component.

It is also an object of the invention to provide a signal processing method to determine the concentration of a material of interest even if noise due to body motion is superposed on the pulse wave signal.

It is also an object of the invention to provide a signal processing method in which noise is reduced from the pulse wave signal, thereby accurately determining a pulse rate even when noise due to motion of the living body has superposed on the pulse wave data signal.

In order to achieve the above objects, according to the invention, there is provided a method of processing observed data, comprising steps of:

receiving a first signal coming from a medium for a predetermined time period as a first data set;

receiving a second signal coming from the medium for the predetermined time period as a second data set;

plotting the first data set and the second data set on a two-dimensional orthogonal coordinate system; and

rotating the first data set and the second data set plotted on the coordinate system by a rotating matrix to separate a signal component and a noise component contained in the observed data.

Preferably, the method further comprises a step of subjecting the signal component to a frequency analysis to determine a fundamental frequency of the signal component.

According to the invention, there is also provided a signal processor, in which the above signal processing method is executed.

In the above configurations, it is enabled signal processing which alleviates a computing burden required for processing two types of signals obtained substantially simultaneously from a single medium to thus extract a common signal component.

According to the invention, there is also provided a pulse photometer adapted to observe a pulse wave of a living body, comprising

a light emitter, adapted to irradiate the living body with a first light beam having a first wavelength and a second light beam having a second wavelength which is different from the first wavelength;

a converter, operable to convert the first light beam and the second light beam, which hav been reflected or transmitted from the living body, into a first data set corresponding to the first wavelength and a second data set corresponding to the second wavelength; and

a processor, operable to process the first data set and the second data set with a rotating matrix to separate a signal component and a noise component contained in the pulse wave.

Preferably, the processor is operable to plot the first data set and the second data set on a two-dimensional orthogonal coordinate system constituted by a first axis corresponding to the first data set and a second axis corresponding to the second data set. Here, the first data set and the second data set plotted on the coordinate system are to be rotated by the rotating matrix.

It is further preferable that a rotating angle of the rotating matrix is determined such that a distribution range of the first data set and the second data set which are projected on one of the first axis and the second axis is minimized.

Preferably, the first data set and the second data set are obtained for a predetermined time period consecutively.

Preferably, the processor is operable to: subject the signal component to a frequency analysis to determine at least one of a fundamental frequency of the pulse wave and a pulse rate of the living body; and obtain a concentration of at least one light-absorbing material in blood of the living body, based on at least one of the fundamental frequency and the pulse rate.

Here, it is preferable that the concentration of the light-absorbing material is at least one of an oxygen saturation in arterial blood, a conc ntration of abnormal hemoglobin in arterial blood, and a concentration of injected dye in arterial blood.

According to the invention, there is also provided a pulse photometer, comprising:

a light emitter, adapted to irradiate a living body with a first light beam having a first wavelength and a second light beam having a second wavelength which is different from the first wavelength;

a converter, operable to convert the first light beam and the second light beam, which have been reflected or transmitted from the living body, into a first data set corresponding to the first wavelength and a second data set corresponding to the second wavelength; and

a processor, operable to: plot the first data set and the second data set on a two-dimensional orthogonal coordinate system corresponding to the first wavelength and the second wavelength; calculate a first norm value for the first data set and a second norm value for the second data set to obtain a norm ratio of the first norm value and the second norm value; and obtain a concentration of at least one light-absorbing material in blood of the living body, based on the norm ratio.

Here, it is preferable that the concentration of the light-absorbing material is at least one of an oxygen saturation in arterial blood, a concentration of abnormal hemoglobin in arterial blood, and a concentration of injected dye in arterial blood.

In the above configurations, it is enabled accurate measurement of the concentration of a material of interest even when noise stemming from motion of the living body has superposed on a pulse wave signal.

Further, even when noise due to motion of the living body has superposed a pulse wave signal, noise is reduced therefrom, so that a stroke and the concentration of a light-absorbing material are accurately determined.

### BRIEF DESCRIPTION OF THE DRAWINGS

The above objects and advantages of the present invention will become more apparent by describing in detail preferred exemplary embodiments thereof with reference to the accompanying drawings, wherein:

FIG. 1 is a block diagram showing a schematic configuration of pulse oximeter which executes a signal processing method of the invention;

FIG. 2 is a graph showing processed data derived from pulse wave signals observed for a predetermined time period;

FIG. 3 is a graph showing detected pulse wave data, in which the amplitude of a red light pulse wave signal is plotted on a vertical axis and the amplitude of an infrared light pulse wave signal is plotted on a horizontal axis;

FIG. 4 is a graph showing that the plotted data shown in FIG. 3 are subjected to a rotating processing;

FIG. 5 is a view showing the waveform of a pulse wave signal processed by a rotating matrix with a rotating angle of 9π/30 [rad];

FIGS. 6A and 6B show spectra of the pulse wave signal before and after the rotating processing is performed;

FIG. 7 is a flowchart showing a processing flow according to a first embodiment of the invention;

FIG. 8 is a flowchart showing a processing flow according to a second embodiment of the invention;

FIGS. 9A and 9B are waveform diagrams for describing the principle of measurement of variations in absorbance of a light-absorbing material in blood;

FIG. 10 is a flowchart showing a processing flow according to a third embodiment of the invention; and

FIG. 11 is a flowchart showing a processing flow according to a fourth embodiment of the invention.

### DETAILED DESCRIPTION OF THE PREFERRED EMBODIMENTS

On the occasion of explanation of an embodiment of the invention, the principle of the invention will be described by taking, as an example, a pulse oximeter for measuring oxygen saturation in arterial blood.

The technique of the invention is not limited to a pulse oximeter, but can also be applied to a pulse photometer which measures abnormal hemoglobin (carboxyhemoglobin, methemoglobin, etc.) and light-absorbing materials in blood, such as dye injected into blood, through use of the principle of pulse photometry.

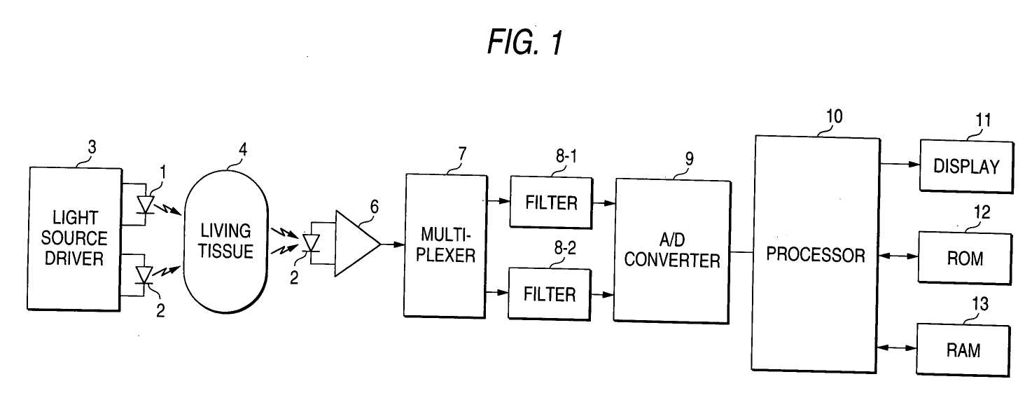

The configuration of a pulse oximeter which measures oxygen saturation in arterial blood is shown in FIG. 1.

Photo emitters 1, 2, which emit light rays of different wavelengths, are activated by a light source driver 3 so as to emit light alternately.

The light adopted for the photo emitters 1, 2 may be embodied by an infrared light (having a wavelength of, e.g., 940 nm) which is less influenced by oxygen saturation in arterial blood, or a red ray (having a wav length of, e.g., 660 nm) which exhibits high sensitivity against a change in oxygen saturation in arterial blood.

The light emitted from the photo emitters 1, 2 passes through living tissue 4 and is received by a photodiode 5 and converted into an electric signal. The reflected light from the living tissue may be used instead of the light passing through living tissue.

The thus-converted signal is amplified by an amplifier 6 and divided into corresponding filters 8-1, 8-2 assigned to respective light wavelengths by a multiplexer 7.

The signals assigned to the filters are filtered through the filters 8-1, 8-2, whereby noise components are reduced and digitized by an A/D converter 9.

The digitized signal trains corresponding to the infrared light and the red light form respective pulse wave signals.

The digitized signal trains are input to a processor 10 and processed in accordance with a program stored in a ROM 12. Oxygen saturation SpO₂ is measured, and a result of measurement is displayed on a display 11.

First, measurement of variations in light absorbance (light attenuation) of a light-absorbing material in blood will be described.

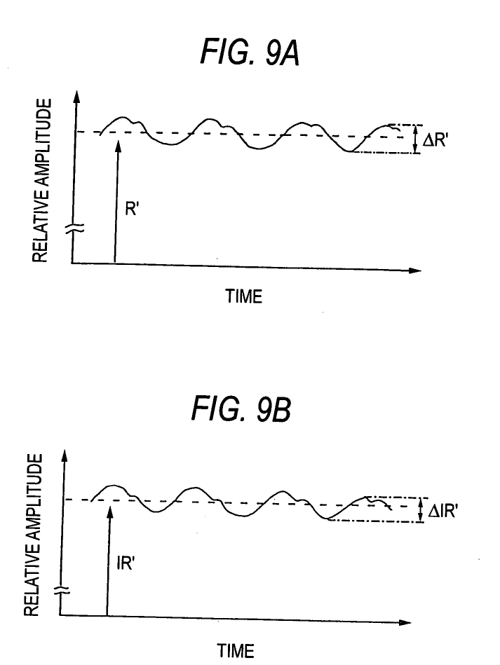

FIG. 9A shows pulse wave data obtained as a result of red light emitted from the photo emitter 1 being received by the photodiode 5 after having passed through the living tissue 4 and the thus-received light being converted into an electric signal. FIG. 9B shows pulse wave data obtained as a result of infrared light emitted from the photo emitter 2 being received by the photodiode 5 after having passed through the living tissue 4 and the thus-received light being converted into an electric signal.

On the assumption that in FIG. 9A the horizontal axis represents time and the vertical axis represents an output of received light, the output of received light produced by the photodiode 5 assumes a waveform pattern into which a DC (direct current) component (R′) and a pulsation component (ΔR′), both belonging to the red light, are superimposed one on the other.

On the assumption that in FIG. 9B the horizontal axis represents time and the vertical axis represents an output of received light, the output of received light produced by the photodiode 5 assumes a waveform pattern into which a DC component (IR′) and a pulsation component (ΔIR′), both belonging to the infrared light, are superimposed one on the other.

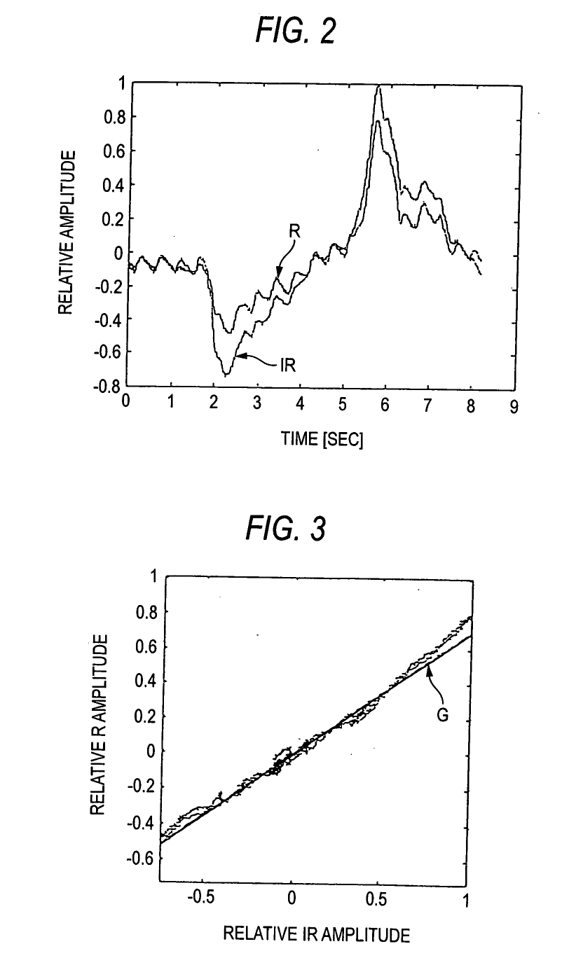

FIG. 2 is a graph plotted by determining a ratio of pulsation components (ΔR′, ΔIR′) to DC components (R′, IR′); that is, (IR=ΔIR′/IR′), in relation to pulse waves such as those shown in FIGS. 9A and 9B, over a period of eight seconds and aligning respective mean values of the obtained amplitude data with zero, as shown in FIG. 2. This alignment operation may be omitted.

Next will be described arithmetic processing for reducing noise in two pulse wave data signals of the two wavelengths digitized by the A/D converter 9 through use of a rotating matrix.

An infrared light and red light are illuminated alternately. Hence, strictly speaking, they are not emitted simultaneously. However, a value of a received infrared light and a value of received red light, being chronologically adjacent to each other, are taken as if they were obtained at the same time. A pulse wave signal of the infrared light for a predetermined time period and a pulse wave signal of the red light for a predetermined time period are plotted on two-dimensional orthogonal coordinates, as shown in FIG. 3.

In FIG. 3, the horizontal axis indicates data pertaining to infrared light IR shown in FIG. 9B and the vertical axis indicates data pertaining to the red light R shown in FIG. 9A.

A ratio of pulsation components to DC components of a pulse wave is determined, to thereby approximate pulsation components of light absorbance attributable to pulsation.

The plotted data in the graph shown in FIG. 3 are not actually on a line angled by 45 degrees from the respective axes. This is because a difference exists between the amplitudes of pulsation components of the infrared light pulse wave and the red light pulse wave, and because noise is superimposed on the pulsation components.

The plotted pulse wave data are subjected to rotational computation through use of a rotating matrix.

A data sequence pertaining to a ratio of pulsation components to DC components of the infrared light pulse wave; i.e., IR, is expressed as follows.

IR={IR(ti):ti=0,1,2,3, . . . } (1)

A data sequence pertaining to a ratio of pulsation components to DC components of the red light pulse wave; i.e., R, is expressed as follows

R={R(ti):ti=0,1,2,3, . . . } (2)

Data pertaining to IR and R, both being obtained at the same time ti, are defined by a matrix in the following manner.

$$S = ( \begin{matrix}IR ( ti ) \\ R ( ti )\end{matrix} ) \qquad ( 3 )$$

Provided that a rotating matrix for effecting rotation by the rotating angle θ [rad] is taken as A, A can be expressed as follows.

$$A = ( \begin{matrix}cos θ & - sin θ \\ sin θ & cos θ\end{matrix} ) \qquad ( 4 \text{-} 1 )$$

The following X is obtained by rotating the pulse wave data S by the rotating angle θ [rad] by the rotating matrix A.

$$X ≡ ( \begin{matrix}X1 ( ti ) \\ X2 ( ti )\end{matrix} ) = A \cdot S = ( \begin{matrix}cos θ & - sin θ \\ sin θ & cos θ\end{matrix} ) ( \begin{matrix}IR ( ti ) \\ R ( ti )\end{matrix} ) \qquad ( 5 )$$

In addition to the rotating matrix A, another rotating matrix A′ provided below may also be employed.

$${A}^{′} = ( \begin{matrix}cos θ & sin θ \\ - sin θ & cos θ\end{matrix} ) \qquad ( 4 \text{-} 2 )$$

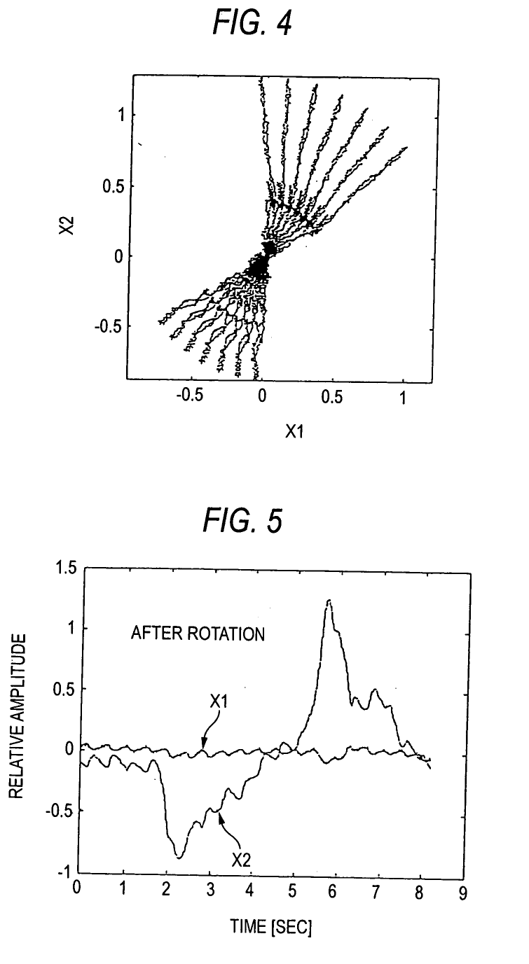

Here, FIG. 4 shows a graph plotted by rotating the pulse wave data S with the rotating angle θ being rotated from 0 to 9π/30 [rad] in increments of π/30 [rad].

As can be seen in FIG. 4, the pulse wave data S are rotated around a point of zero for the horizontal and vertical axes (i.e., a point where a mean value of the red light pulse wave and a mean value of the infrared light pulse wave are achieved). When θ is 9π/30 [rad], the range in which the data projected onto the horizontal axis (X1) are distributed is minimized, and the range in which the data projected onto the vertical axis (X2) are distributed is maximized.

When θ is rotated from 9π/30 [rad] by further π/2 [rad] up to 24π/30 [rad](=12π/15 [rad]), the range in which the data projected onto the horizontal axis (X1) are distributed is obviously maximized, and the range in which the data projected onto the vertical axis (X2) are distributed is obviously minimized.

There will now be described the kind of waveform obtained as a result of the pulse waveform data S being processed into X by the rotating matrix A achieved when θ is rotated to 9π/30 [rad] and 24π/30 [rad].

FIG. 5 shows a waveform of X obtained by processing the pulse wave data S shown in FIG. 2 through use of the rotating matrix A with the rotating angle θ being taken as 9π/30 [rad].

X1(ti) at which the range projected on the horizontal axis has been minimized is computed by the following equation.

X1(ti)[θ=9π/30]=cos θ·IR(ti)−sin θ·R(ti) (6)

X2(ti) at which the range projected on the horizontal axis has been maximized is computed by the following equation.

X2(ti)[θ=9π/30]=sin θ·IR(ti)+cos θ·R(ti) (7)

Noise is understood to be reduced from the wave form of X1 shown in FIG. 5.

When the pulse wave data S are processed by the rotating matrix A with θ being taken as 24π/30 [rad], the waveform of X2 becomes another waveform from which noise has been reduced.

X1(ti) at which the range projected on the horizontal axis is maximized is computed by the following equation.

X1(ti)[θ=24π/30]=cos θ·IR(ti)−sin θ·R(ti) (8)

X2(ti) at which the range projected on the vertical axis is minimized is computed by the following equation.

X2(ti)[θ=24π/30]=sin θ·IR(ti)+cos θ·R(ti) (9)

Thus, the rotating angle θ is determined such that the range in which the data projected on the horizontal axis are distributed is minimized. Processing the pulse wave data S with the thus determined rotating angle, there can be obtained a principal component waveform of a pulse wave whose noise is suppressed.

Next, computation of the fundamental frequency of a pulse wave will be described.

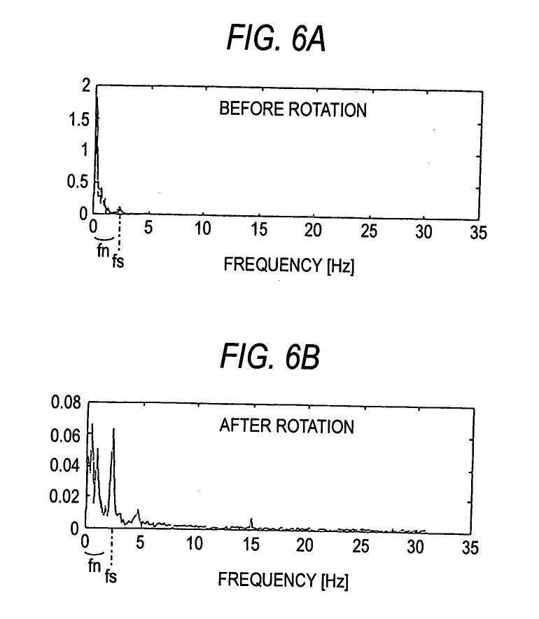

FIG. 6A shows a spectrum of a pulse wave signal from which noise has not been reduced (corresponding to FIG. 2). FIG. 6B shows a spectrum of a principal component waveform from which noise has been reduced by use of the rotating matrix. These spectra are obtained by frequency analysis. The horizontal axis represents a frequency, and the vertical axis shows a spectrum.

In relation to a spectrum of a pulse wave signal obtained before noise is reduced. Before the rotation, as shown in FIG. 6A, a spectrum in a noise frequency range fn appears intensively, whereas a spectrum in the fundamental frequency fs of the pulse wave signal is substantially absent.

In relation to a spectrum obtained by frequency analysis of a principal component waveform of pulse wave whose noise has been reduced through use of the rotating matrix. After the rotation, as shown in FIG. 6B, a spectrum in the fundamental frequency fs of the pulse wave signal is seen to intensively appear so as to be distinguishable from a spectrum in the noise frequency band fn. The fundamental frequency fs of the pulse wave signal can be determined.

If the fundamental frequency fs [HZ] of the pulse wave signal is determined, a pulse rate (60 fs [times/min.]) can be readily determined.

As mentioned above, the principal component waveform of pulse wave whose noise has been reduced can be obtained through use of a rotating matrix of predetermined angle. The fundamental frequency or pulse rate of the pulse wave signal can be determined.

Here, the rotating angle may be determined beforehand or changed adaptively during a period of measurement.

FIG. 3 is a graph formed when the red light pulse wave data R are plotted on the vertical axis and the infrared light pulse wave data IR are plotted on the horizontal axis. The gradient G of the graph is determined through use of a norm ratio.

First, the L2 norm (square norm) for the infrared pulse wave. data IR is determined. Since an infrared light pulse wave data sequence is determined by Equation 1, the L2 norm can be expressed by the following equation.

∥IR∥=\sqrt{ΣIR(ti)²} (10)

Next, the L2 norm of the red light pulse wave data R is determined. Since a red light pulse wave data sequence is determined by Equation 2, the L2 norm can be expressed by the following equation.

∥R∥={square root over ((ΣR(ti)²)} (11)

Here, provided that

$$Φ = \frac{‖ R ‖}{‖ IR ‖} , \qquad ( 12 )$$

Φ correlates with the oxygen saturation SpO₂. Taking a function representing the correlation as “f,” the oxygen saturation will be expressed as follows.

SpO₂=f(Φ) (13)

Thus, the oxygen saturation SpO₂ can be determined. FIG. 3 shows a line whos gradient is determined by a norm ratio.

Here, the term “norm” refers to a mathematical concept. An Euclidean norm or a square norm maps onto a scalar the magnitude of a vector having “n” elements. As mentioned above, the oxygen saturation SpO2 can be determined on the basis of a ratio of the L2 norm value (square norm) of the red light pulse wave data R over a predetermined time period and the L2 norm value of the infrared light pulse wave data over a predetermined time period.

Here, the red light pulse wave data R and the infrared light pulse wave data IR over a predetermined time period may be used for a given time period in reverse chronological order from the sequentially-obtained present pulse wave.

The L2 norm is used for the norm value, but another norm value determined by another computing method may also be used.

The oxygen saturation may be preferably computed with the above explained norm ratio in a case where the noise component is relatively small with respect to the pulse wave signal. On the other hand, in a case where the noise component is relatively large with respect to the pulse wave signal,

In relation to computation of the oxygen saturation, the oxygen saturation may be computed with a fundamental frequency obtained by the above explained rotating computation, in place of a fundamental frequency obtained by the frequency analysis disclosed in Japanese Patent Publication No. 2003-135434A.

The apparatus using the foregoing principle will now be described by reference to FIGS. 1, 7 through 11.

As described previously, the photo emitt rs 1, 2 are activated by the light source driver 3 so as to alternately effect emission, thereby emitting light rays of different wavelengths. The light rays emitted from the photo emitters 1, 2 pass through the living tissue 4 and are then received by the photodiode 5, where the light is converted into an electric signal. The thus-converted signals are amplified by the amplifier 6 and divided to the filters 8-1, 8-2 assigned to the respective light wavelengths, by the multiplexer 7. The signals allocated to the respective filters are filtered by the filters. 8-1, 8-2, whereby noise components of the signals are reduced. The signals are digitized by the A/D converter 9. The digitized signal trains corresponding to the infrared light and the red light form the pulse waves. The digitized signal trains are input to the processor 10 and processed by a program stored in the ROM 12, wherein a pulse rate PR and oxygen saturation SpO₂ are computed. The resultant computed value is displayed on the display 11.

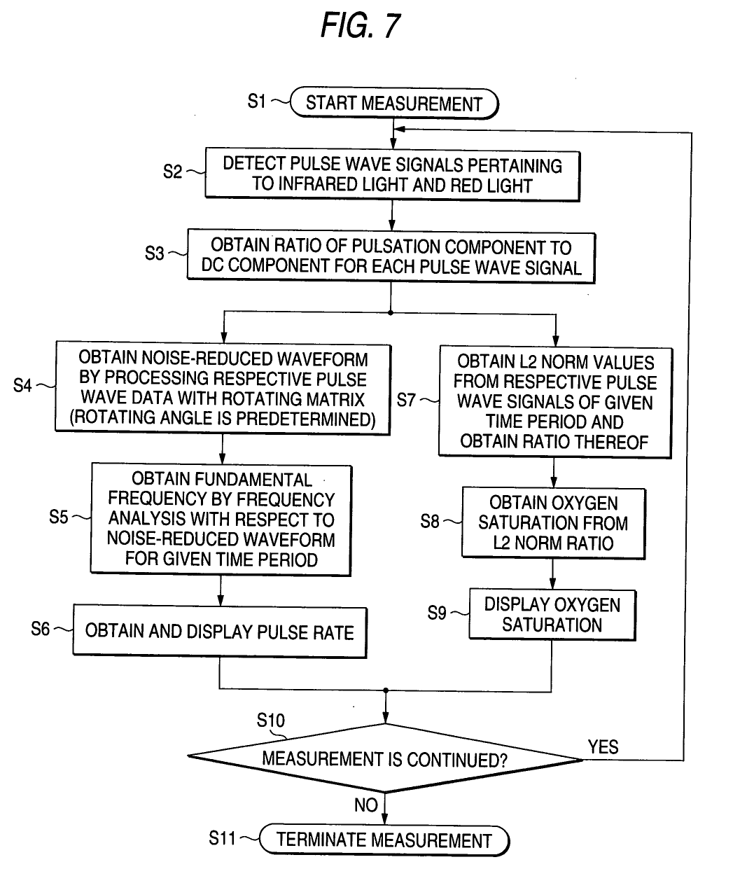

As a first embodiment of the invention, a processing flow to be used for computing the pulse rate PR and the oxygen saturation SpO₂ are described by reference to FIG. 7. Measurement is then initiated (step S1). The red light pulse wave and the infrared light pulse wave are detected in the manner mentioned above (step S2). The digitized signal trains (respective pulse wave data sets) are acquired by the processor 10.

In accordance with the program stored in the ROM 12, the processor 10 processes the pulse wave data in the following manner by reading and writing data, which are being processed, from and to a RAM 13.

First, a pulsation component ratio of the infrared light pulse wave to a DC component of the pulse wave and a pulsation component ratio of the red light pulse wave to a DC component of th pulse wave are determined (step S3).

Next, processing for determining the pulse rate PR (steps S4 to S6) and processing for determining oxygen saturation SpO₂ (steps S7 to S9) are performed simultaneously.

Through the processing for determining the pulse rate PR (steps S4 to S6), a waveform whose noise is reduced is obtained from the data S pertaining to the infrared light pulse wave data IR and the red light pulse wave data R, according to Equation 5 by the rotating matrix A for which a rotating angle is set beforehand (step S4).

Here, the rotation angle to be set is such an angle that a range on one of the axes shown in FIG. 4 on which the data plotted as shown in FIG. 3 are projected and distributed is minimized. The rotating angle may be, for example, 9π/30 [rad] or 24π/30 [rad]. The waveform whose noise has been reduced can be obtained from the data pertaining to an axial component at which the distribution range of the projected data is minimized.

The waveform whose noise has been reduced is subjected to frequency analysis in such a manner as shown in FIG. 6B, thereby determining the fundamental frequency of the pulse wave data (step S5).

The pulse rate is determined from the fundamental frequency according to 60 fs [times/min] and displayed on the display 11.

During processing for determining oxygen saturation SpO₂ (steps S7 to S9), the L2 norm values are determined from the infrared light pulse wave data IR and the red light pulse wave data R, both being obtained over a predetermined time period, by Equations (10) and (11). A ratio between the both L2 norm values is det rmined by Equation (12).

A ratio of the infrared light pulse signal whose noise has been reduced to the red light pulse signal whose noise has been reduced is determined, to thus compute oxygen saturation (step S7). The L2 norm ratio is taken as Φ, the oxygen saturation SpO₂ is determined according to Equation (12) (step S8), and the thus-obtained oxygen saturation is displayed on the display 11 (step S9).

When measurement. is continued, processing returns to step S2, where processing is iterated. When measurement is not performed, measurement is completed (step S11).

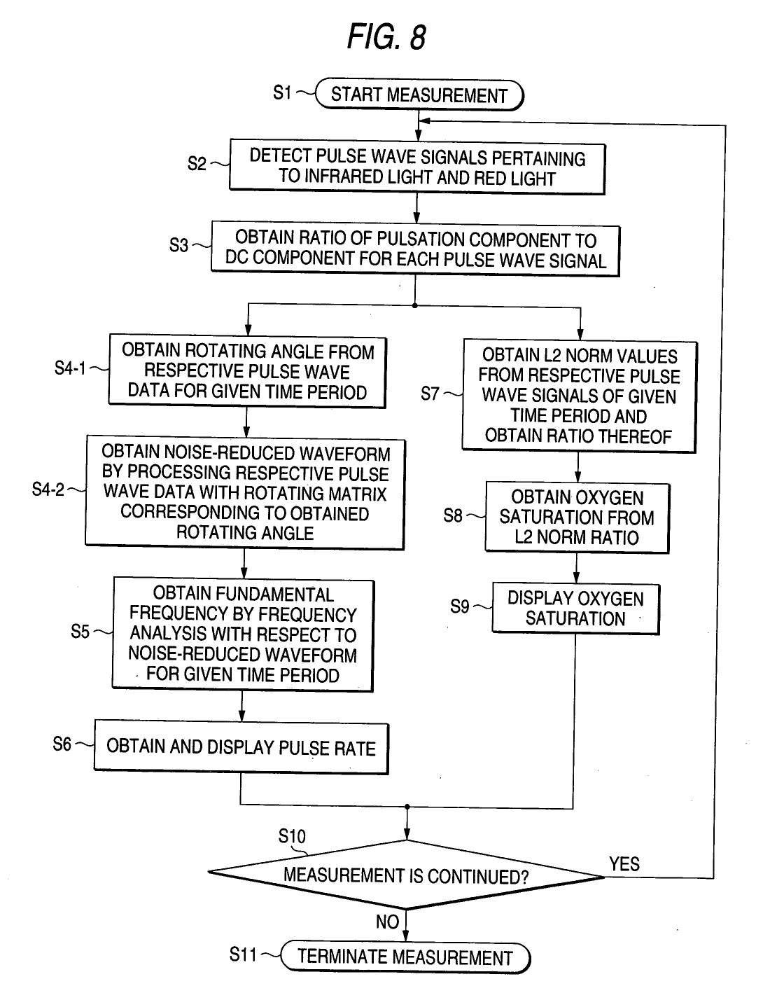

Next, a second embodiment of the invention will be described by reference to FIG. 8.

A difference between the first and second embodiments lies in that, in step S4, a rotating angle is not determined beforehand but is determined from obtained data. As shown in FIG. 8, processing is performed with step S4-1 being separated from step S4-2. The other steps are the same as those of the first embodiment, and hence their repeated explanations are omitted.

During processing (steps S4 to S6) for determining a pulse rate PR, a graph such as that shown in FIG. 3 is first plotted through use of the infrared light pulse wave data IR and the red light pulse wave data R, both being obtained over a given time period.

Then, a rotating operation is performed with respect to the plotted data to find out a rotating angle at which a distribution range of the data projected on one of axes shown in FIG. 4 is minimized (step S4-1). Next, pulse wave data of respective wavelengths are processed by a rotating matrix through the thus obtained rotating angle. Th waveform whos noise has been reduced can be obtained from the data pertaining to an axial component at which the distribution range of the projected data is minimized (step S4-2).

As mentioned above, the characteristic of the second embodiment lies in that the rotating angle of the rotating matrix is not a fixed angle and has an adaptive characteristic such that the rotating angle is variable, as necessary, according to detected pulse wave data.

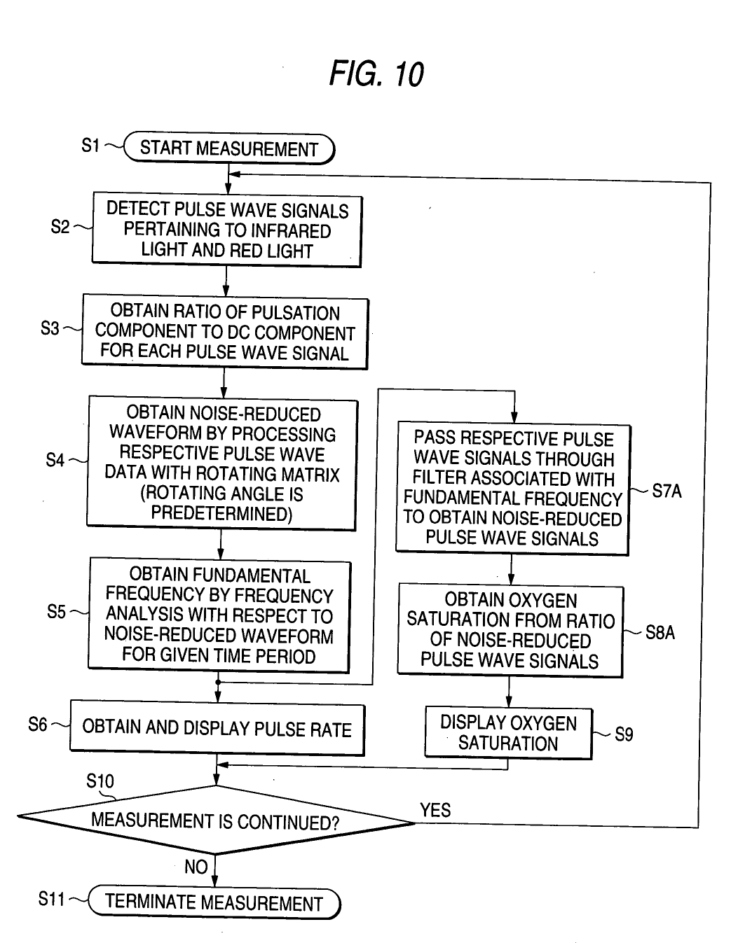

As a third embodiment of the invention, the pulse rate PR and the oxygen saturation SpO₂ are replaced with the fundamental frequency determined by use of frequency analysis. By reference to FIG. 10, the processing flow, which performs processing through use of the fundamental frequency determined by the rotational processing, will be described. The steps as same as those of the first embodiment are designated by the same reference numerals, and their repeated explanations are omitted.

During processing for determining oxygen saturation SpO₂ (steps S7A to S9), in this embodiment, a noise-reduced signal is obtained by causing the infrared light pulse wave signal and the red light pulse wave signal to pass through a filter formed from the fundamental frequency (obtained by step S5) or from combination of the fundamental frequency and a harmonic wave thereof (step S7A).

A ratio of the infrared light pulse signal whose noise has been reduced to the red light pulse signal whose noise has been reduced is determined, to thus compute oxygen saturation (step S8A), and the computed oxygen saturation is displayed on the display 11 (step S9).

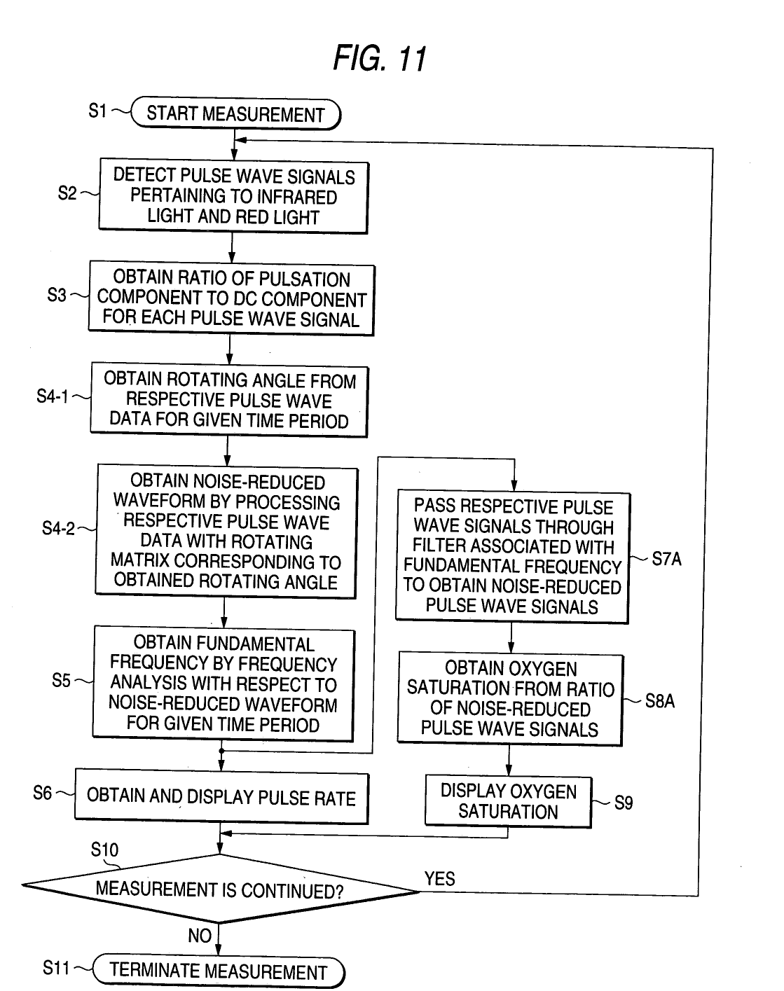

Next, a fourth embodiment of the invention will be described by reference to FIG. 11.

A difference between the third and fourth embodiments lies in that, in step S4, a rotating angle is not determined beforehand and that a rotating angle is determined from obtained data. As shown in FIG. 11, processing is performed with step S4-1 being separated from step S4-2. The other steps are the same as those of the third embodiment, and hence their repeated explanations are omitted.

During process (steps S4-1 to S6) for determining a pulse rate PR, a graph such as that shown in FIG. 3 is first plotted through use of the infrared light pulse wave data IR and the red light pulse wave data R, both being obtained over a given time period. Then, a rotating operation is performed with respect to the plotted data to find out a rotating angle at which a distribution range of the data projected on one of axes shown in FIG. 4 is minimized (step S4-1). Next, pulse wave data of respective wavelengths are processed by a rotating matrix through then thus obtained rotating angle. The waveform whose noise has been reduced can be obtained from the data pertaining to an axial component at which the distribution range of the projected data is minimized (step S4-2).

As mentioned above, the characteristic of the fourth embodiment lies in that the rotating angle of the rotating matrix is not a fixed angle and has an adaptive characteristic such that the rotating angle is variable, as necessary, according to detected pulse wave data.

The foregoing descriptions have described the invention by taking, as an example, a pulse oximeter which measures oxygen saturation in arterial blood. The technique of the invention is not limited to a pulse oximeter and can also be applied to an apparatus (pulse photometer), which measures abnormal hemoglobin (carboxyhemoglobin, methemoglobin, etc.) and light-absorbing materials in blood, such as dye injected into blood, through use of the principle of pulse photometry, by selection of a wavelength of the light source.

Although the present invention has been shown and described with reference to specific preferred embodiments, various changes and modifications will be apparent to those skilled in the art from the teachings herein. Such changes and modifications as are obvious are deemed to come within the spirit, scope and contemplation of the invention as defined in the appended claims.

What is claimed is:

1. A method of processing observed data, comprising steps of:
receiving a first signal coming from a medium for a predetermined time period as a first data set;
receiving a second signal coming from the medium for the predetermined time period as a second data set;
plotting the first data set and the second data set on a two-dimensional orthogonal coordinate system; and
rotating the first data set and the second data set plotted on the coordinate system by a rotating matrix to separate a signal component and a noise component contained in the observed data.

2. The signal processing method as set forth in claim 1, further comprising a step of subjecting the signal component to a frequency analysis to determine a fundamental frequency of the signal component.

3. A signal processor, in which the signal processing method as set forth in claim 1 is executed.

4. A pulse photometer adapted to observe a pulse wave of a living body, comprising
a light emitter, adapted to irradiate the living body with a first light beam having a first wavelength and a second light beam having a second wavelength which is different from the first wavelength;
a converter, operable to convert the first light beam and the second light beam, which have been reflected or transmitted from the living body, into a first data set corresponding to the first wavelength and a second data set corresponding to the second wavelength; and
a processor, operable to process the first data set and the second data set with a rotating matrix to separate a signal component and a noise component contained in the pulse wave.

5. The pulse photometer as set forth in claim 4, wherein:
the processor is operable to plot the first data set and the second data set on a two-dimensional orthogonal coordinate system constituted by a first axis corresponding to the first data set and a second axis corresponding to the second data set; and
the first data set and the second data set plotted on the coordinate system are to be rotated by the rotating matrix.

6. The pulse photometer as set forth in claim 4, wherein the first data set and the second data set are obtained for a predetermined time period consecutively.

7. The pulse photometer as set forth in claim 5, wherein a rotating angle of the rotating matrix is determined such that a distribution range of the first data set and the second data set which are projected on one of the first axis and the second axis is minimized.

8. A pulse photometer, comprising
a light emitter, adapted to irradiate a living body with a first light beam having a first wavelength and a second light beam having a second wavelength which is different from the first wavelength;
a converter, operable to convert the first light beam and the second light beam, which have been reflected or transmitted from the living body, into a first data set corresponding to the first wavelength and a second data set corresponding to the second wavelength; and
a processor, operable to:
plot the first data set and the second data set on a two-dimensional orthogonal coordinate system corresponding to the first wavelength and the second wavelength;
calculate a first norm value for the first data set and a second norm value for the second data set to obtain a norm ratio of the first norm value and the second norm value; and
obtain a concentration of at least one light-absorbing material in blood of the living body, based on the norm ratio.

9. The pulse photometer as set forth in claim 8, wherein the concentration of the light-absorbing material is at least one of an oxygen saturation in arterial blood, a concentration of abnormal hemoglobin in arterial blood, and a concentration of injected dye in arterial blood.

10. The pulse photometer as set forth in claim 4, wherein the processor is operable to:
subject the signal component to a frequency analysis to determine at least one of a fundamental frequency of the pulse wave and a pulse rate of the living body; and
obtain a concentration of at least one light-absorbing material in blood of the living body, based on at least one of the fundamental frequency and the pulse rate.

11. The pulse photometer as set forth in claim 10, wherein the concentration of the light-absorbing material is at least one of an oxygen saturation in arterial blood, a concentration of abnormal hemoglobin in arterial blood, and a concentration of injected dye in arterial blood.
